(19) United States
(12) Patent Application Publication
(10) Pub. No.: US 2005/0074752 A1
(43) Pub. Date: Apr. 7, 2005

(54) Synthetic hepatitis C genes

(75) Inventors: John J. Donnelly, Moraga, CA (US); Margaret A. Liu, Lafayette, CA (US); John W. Shiver, Chalfont, PA (US); Tong-Ming Fu, Ambler, PA (US)

Correspondence Address: MERCK AND CO., INC, P O BOX 2000, RAHWAY, NJ, 07065-0907, US

(73) Assignee: Merck & Co., Inc., Rahway, NJ (US)

(21) Appl. No.: 10/664,391

(22) Filed: Sep. 17, 2003

Related U.S. Application Data
(63) Continuation of application No. 09/194,949, filed on Feb. 17, 2000, now Pat. No. 6,653,125.
(60) Provisional application No. 60/020,494, filed on Jun. 11, 1996.
(60) Provisional application No. 60/033,534, filed on Dec. 20, 1996.

(51) Int. Cl. C12Q 1/70; C07H 21/04; C12N 7/01; C07K 14/02; C12N 15/86

(52) U.S. Cl. 435/5; 435/69.3; 435/235.1; 435/456; 435/325; 530/350; 536/23.72

(57) ABSTRACT

This invention relates to novel formulations of pharmaceutical products, specifically nucleic acid vaccine products. The nucleic acid vaccine products, when introduced directly into muscle cells, induce the production of immune responses which specifically recognize Hepatitis C virus (HCV).

### CROSS-REFERENCE TO RELATED APPLICATIONS

Not applicable.

### STATEMENT REGARDING FEDERALLY-SPONSORED R&D

Not applicable.

### REFERENCE TO MICROFICHE APPENDIX

Not applicable.

### FIELD OF THE INVENTION

Not applicable.

### BACKGROUND OF THE INVENTION

This invention relates to novel nucleic acid pharmaceutical products, specifically nucleic acid vaccine products. The nucleic acid vaccine products, when introduced directly into muscle cells, induce the production of immune responses which specifically recognize Hepatitis C virus (HCV).

Hepatitis C Virus

Non-A, Non-B hepatitis (NANBH) is a transmissible disease (or family of diseases) that is believed to be virally induced, and is distinguishable from other forms of virus-associated liver disease, such as those caused by hepatitis A virus (HAV), hepatitis B virus (HBV), delta hepatitis virus (HDV), cytomegalovirus (CMV) or Epstein-Barr virus (EBV). Epidemiologic evidence suggests that there may be three types of NANBH: the water-borne epidemic type; the blood or needle associated type; and the sporadically occurring (community acquired) type. However, the number of causative agents is unknown. Recently, a new viral species, hepatitis C virus (HCV) has been identified as the primary (if not only) cause of blood-associated NANBH (BB-NANBH). Hepatitis C appears to be the major form of transfusion-associated hepatitis in a number of countries, including the United States and Japan. There is also evidence implicating HCV in induction of hepatocellular carcinoma. Thus, a need exists for an effective method for preventing or treating HCV infection: currently, there is none.

The HCV may be distantly related to the flaviviridae. The Flavivirus family contains a large number of viruses which are small, enveloped pathogens of man. The morphology and composition of Flavivirus particles are known, and are discussed in M. A. Brinton, in “The Viruses: The Togaviridae And Flaviviridae” (Series eds. Fraenkel-Conrat and Wagner, vol. eds. Schlesinger and Schlesinger, Plenum Press, 1996), pp. 327-374. Generally, with respect to morphology, Flaviviruses contain a central nucleocapsid surrounded by a lipid bilayer. Virions are spherical and have a diameter of about 40-50 nm. Their cores are about 25-30 nm in diameter. Along the outer surface of the virion envelope are projections measuring about 5-10 nm in length with terminal knobs about 2 nm in diameter. Typical examples of the family include Yellow Fever virus, West Nile virus, and Dengue Fever virus. They possess positive-stranded RNA genomes (about 11,000 nucleotides) that are slightly larger than that of HCV and encode a polyprotein precursor of about 3500 amino acids. Individual viral proteins are cleaved from this precursor polypeptide.

The genome of HCV appears to be single-stranded RNA containing about 10,000 nucleotides. The genome is positive-stranded, and possesses a continuous translational open reading frame (ORF) that encodes a polyprotein of about 3,000 amino acids. In the ORF, the structural proteins appear to be encoded in approximately the first quarter of the N-terminal region, with the majority of the polyprotein attributed to non-structural proteins. When compared with all known viral sequences, small but significant co-linear homologies are observed with the nonstructural proteins of the Flavivirus family, and with the pestiviruses (which are now also considered to be part of the Flavivirus family).

Intramuscular inoculation of polynucleotide constructs, i.e., DNA plasmids encoding proteins have been shown to result in the in situ generation of the protein in muscle cells. By using cDNA plasmids encoding viral proteins, both antibody and CTL responses were generated, providing homologous and heterologous protection against subsequent challenge with either the homologous or cross-strain protection, respectively. Each of these types of immune responses offers a potential advantage over existing vaccination strategies. The use of PNVs (polynucleotide vaccines) to generate antibodies may result in an increased duration of the antibody responses as well as the provision of an antigen that can have both the exact sequence of the clinically circulating strain of virus as well as the proper post-translational modifications and conformation of the native protein (vs. a recombinant protein). The generation of CTL responses by this means offers the benefits of cross-strain protection without the use of a live potentially pathogenic vector or attenuated virus.

Therefore, this invention contemplates methods for introducing nucleic acids into living tissue to induce expression of proteins. The invention provides a method for introducing viral proteins into the antigen processing pathway to generate virus-specific immune responses including, but not limited to, CTLs. Thus, the need for specific therapeutic agents capable of eliciting desired prophylactic immune responses against viral pathogens is met for HCV virus by this invention. Of particular importance in this therapeutic approach is the ability to induce T-cell immune responses which can prevent infections even of virus strains which are heterologous to the strain from which the antigen gene was obtained. Therefore, this invention provides DNA constructs encoding viral proteins of the hepatitis C virus core, envelope (E1), nonstructural (NS5) genes or any other HCV genes which encode products which generate specific immune responses including but not limited to CTLs.

DNA Vaccines

Benvenisty, N., and Reshef, L. [PNAS 83, 9551-9555, (1996)] showed that CaCl₂-precipitated DNA introduced into mice intraperitoneally (i.p.), intravenously (i.v.) or intramuscularly (i.m.) could be expressed. The i.m. injection of DNA expression vectors without CaCl₂ treatment in mice resulted in the uptake of DNA by the muscle cells and expression of the protein encoded by the DNA. The plasmids were maintained episomally and did not replicate. Subsequently, persistent expression has been observed after i.m. injection in skeletal muscle of rats, fish and primates, and cardiac muscle of rats. The technique of using nucleic acids as therapeutic agents was reported in WO90/11092 (4 Oct. 1990), in which polynucleotides were used to vaccinate vertebrates.

It is not necessary for the success of the method that immunization be intramuscular. The introduction of gold microprojectiles coated with DNA encoding bovine growth hormone (BGH) into the skin of mice resulted in production of anti-BGH antibodies in the mice. A jet injector has been used to transfect skin, muscle, fat, and mammary tissues of living animals. Various methods for introducing nucleic acids have been reviewed. Intravenous injection of a DNA:cationic liposome complex in mice was shown by Zhu et al., [Science 261:209-211 (9 Jul. 1993) to result in systemic expression of a cloned transgene. Ulmer et al., [Science 259:1745-1749, (1993)] reported on the heterologous protection against influenza virus infection by intramuscular injection of DNA encoding influenza virus proteins.

The need for specific therapeutic and prophylactic agents capable of eliciting desired immune responses against pathogens and tumor antigens is met by the instant invention. Of particular importance in this therapeutic approach is the ability to induce T-cell immune responses which can prevent infections or disease caused even by virus strains which are heterologous to the strain from which the antigen gene was obtained. This is of particular concern when dealing with HIV as this virus has been recognized to mutate rapidly and many virulent isolates have been identified [see, for example, LaRosa et al., Science 249:932-935 (1990), identifying 245 separate HIV isolates]. In response to this recognized diversity, researchers have attempted to generate CTLs based on peptide immunization. Thus, Takahashi et al., [Science 255:333-336 (1992)] reported on the induction of broadly cross-reactive cytotoxic T cells recognizing an HIV envelope (gp160) determinant. However, those workers recognized the difficulty in achieving a truly cross-reactive CTL response and suggested that there is a dichotomy between the priming or restimulation of T cells, which is very stringent, and the elicitation of effector function, including cytotoxicity, from already stimulated CTLs.

Wang et al. reported on elicitation of immune responses in mice against HIV by intramuscular inoculation with a cloned, genomic (unspliced) HIV gene. However, the level of immune responses achieved in these studies was very low. In addition, the Wang et al., DNA construct utilized an essentially genomic piece of HIV encoding contiguous Tat/REV-gp160-Tat/REV coding sequences. As is described in detail below, this is a suboptimal system for obtaining high-level expression of the gp160. It also is potentially dangerous because expression of Tat contributes to the progression of Karposi's Sarcoma.

WO 93/17706 describes a method for vaccinating an animal against a virus, wherein carrier particles were coated with a gene construct and the coated particles are accelerated into cells of an animal.

The instant invention contemplates any of the known methods for introducing polynucleotides into living tissue to induce expression of proteins. However, this invention provides a novel immunogen for introducing proteins into the antigen processing pathway to efficiently generate specific CTLs and antibodies.

Codon Usage and Codon Context

The codon pairings of organisms are highly nonrandom, and differ from organism to organism. This information is used to construct and express altered or synthetic genes having desired levels of translational efficiency, to determine which regions in a genome are protein coding regions, to introduce translational pause sites into heterologous genes, and to ascertain relationship or ancestral origin of nucleotide sequences.

The expression of foreign heterologous genes in transformed organisms is now commonplace. A large number of mammalian genes, including, for example, murine and human genes, have been successfully inserted into single celled organisms. Standard techniques in this regard include introduction of the foreign gene to be expressed into a vector such as a plasmid or a phage and utilizing that vector to insert the gene into an organism. The native promoters for such genes are commonly replaced with strong promoters compatible with the host into which the gene is inserted. Protein sequencing machinery permits elucidation of the amino acid sequences of even minute quantities of native protein. From these amino acid sequences, DNA sequences coding for those proteins can be inferred. DNA synthesis is also a rapidly developing art, and synthetic genes corresponding to those inferred DNA sequences can be readily constructed.

Despite the burgeoning knowledge of expression systems and recombinant DNA, significant obstacles remain when one attempts to express a foreign or synthetic gene in an organism. Many native, active proteins, for example, are glycosylated in a manner different from that which occurs when they are expressed in a foreign host. For this reason, eukaryotic hosts such as yeast may be preferred to bacterial hosts for expressing many mammalian genes. The glycosylation problem is the subject of continuing research.

Another problem is more poorly understood. Often translation of a synthetic gene, even when coupled with a strong promoter, proceeds much less efficiently than would be expected. The same is frequently true of exogenous genes foreign to the expression organism. Even when the gene is transcribed in a sufficiently efficient manner that recoverable quantities of the translation product are produced, the protein is often inactive or otherwise different in properties from the native protein.

It is recognized that the latter problem is commonly due to differences in protein folding in various organisms. The solution to this problem has been elusive, and the mechanisms controlling protein folding are poorly understood.

The problems related to translational efficiency are believed to be related to codon context effects. The protein coding regions of genes in all organisms are subject to a wide variety of functional constraints, some of which depend on the requirement for encoding a properly functioning protein, as well as appropriate translational start and stop signals. However, several features of protein coding regions have been discerned which are not readily understood in terms of these constraints. Two important classes of such features are those involving codon usage and codon context.

It is known that codon utilization is highly biased and varies considerably between different organisms. Codon usage patterns have been shown to be related to the relative abundance of tRNA isoacceptors. Genes encoding proteins of high versus low abundance show differences in their codon preferences. The possibility that biases in codon usage alter peptide elongation rates has been widely discussed. While differences in codon use are associated with differences in translation rates, direct effects of codon choice on translation have been difficult to demonstrate. Other proposed constraints on codon usage patterns include maximizing the fidelity of translation and optimizing the kinetic efficiency of protein synthesis.

Apart from the non-random use of codons, considerable evidence has accumulated that codon/anticodon recognition is influenced by sequences outside the codon itself, a phenomenon termed “codon context.” There exists a strong influence of nearby nucleotides on the efficiency of suppression of nonsense codons as well as missense codons. Clearly, the abundance of suppressor activity in natural bacterial populations, as well as the use of “termination” codons to encode selenocysteine and phosphoserine require that termination be context-dependent. Similar context effects have been shown to influence the fidelity of translation, as well as the efficiency of translation initiation.

Statistical analyses of protein coding regions of E. coli have demonstrate another manifestation of “codon context.” The presence of a particular codon at one position strongly influences the frequency of occurrence of certain nucleotides in neighboring codons, and these context constraints differ markedly for genes expressed at high versus low levels. Although the context effect has been recognized, the predictive value of the statistical rules relating to preferred nucleotides adjacent to codons is relatively low. This has limited the utility of such nucleotide preference data for selecting codons to effect desired levels of translational efficiency.

The advent of automated nucleotide sequencing equipment has made available large quantities of sequence data for a wide variety of organisms. Understanding those data presents substantial difficulties. For example, it is important to identify the coding regions of the genome in order to relate the genetic sequence data to protein sequences. In addition, the ancestry of the genome of certain organisms is of substantial interest. It is known that genomes of some organisms are of mixed ancestry. Some sequences that are viral in origin are now stably incorporated into the genome of eukaryotic organisms. The viral sequences themselves may have originated in another substantially unrelated species. An understanding of the ancestry of a gene can be important in drawing proper analogies between related genes and their translation products in other organisms.

There is a need for a better understanding of codon context effects on translation, and for a method for determining the appropriate codons for any desired translational effect. There is also a need for a method for identifying coding regions of the genome from nucleotide sequence data. There is also a need for a method for controlling protein folding and for insuring that a foreign gene will fold appropriately when expressed in a host. Genes altered or constructed in accordance with desired translational efficiencies would be of significant worth.

Another aspect of the practice of recombinant DNA techniques for the expression by microorganisms of proteins of industrial and pharmaceutical interest is the phenomenon of “codon preference”. While it was earlier noted that the existing machinery for gene expression is genetically transformed host cells will “operate” to construct a given desired product, levels of expression attained in a microorganism can be subject to wide variation, depending in part on specific alternative forms of the amino acid-specifying genetic code present in an inserted exogenous gene. A “triplet” codon of four possible nucleotide bases can exist in 64 variant forms. That these forms provide the message for only 20 different amino acids (as well as transcription initiation and termination) means that some amino acids can be coded for by more than one codon. Indeed, some amino acids have as many as six “redundant”, alternative codons while some others have a single, required codon. For reasons not completely understood, alternative codons are not at all uniformly present in the endogenous DNA of differing types of cells and there appears to exist a variable natural hierarchy or “preference” for certain codons in certain types of cells.

As one example, the amino acid leucine is specified by any of six DNA codons including CTA, CTC, CTG, CTT, TTA, and TTG (which correspond, respectively, to the mRNA codons, CUA, CUC, CUG, CUU, UUA and UUG). Exhaustive analysis of genome codon frequencies for microorganisms has revealed endogenous DNA of E. coli most commonly contains the CTG leucine-specifying codon, while the DNA of yeasts and slime molds most commonly includes a TTA leucine-specifying codon. In view of this hierarchy, it is generally held that the likelihood of obtaining high levels of expression of a leucine-rich polypeptide by an E. coli host will depend to some extent on the frequency of codon use. For example, a gene rich in TTA codons will in all probability be poorly expressed in E. coli, whereas a CTG rich gene will probably highly express the polypeptide. Similarly, when yeast cells are the projected transformation host cells for expression of a leucine-rich polypeptide, a preferred codon for use in an inserted DNA would be TTA.

The implications of codon preference phenomena on recombinant DNA techniques are manifest, and the phenomenon may serve to explain many prior failures to achieve high expression levels of exogenous genes in successfully transformed host organisms-a less “preferred” codon may be repeatedly present in the inserted gene and the host cell machinery for expression may not operate as efficiently. This phenomenon suggests that synthetic genes which have been designed to include a projected host cell's preferred codons provide a preferred form of foreign genetic material for practice of recombinant DNA techniques.

Protein Trafficking

The diversity of function that typifies eukaryotic cells depends upon the structural differentiation of their membrane boundaries. To generate and maintain these structures, proteins must be transported from their site of synthesis in the endoplasmic reticulum to predetermined destinations throughout the cell. This requires that the trafficking proteins display sorting signals that are recognized by the molecular machinery responsible for route selection located at the access points to the main trafficking pathways. Sorting decisions for most proteins need to be made only once as they traverse their biosynthetic pathways since their final destination, the cellular location at which they perform their function, becomes their permanent residence.

Maintenance of intracellular integrity depends in part on the selective sorting and accurate transport of proteins to their correct destinations. Over the past few years the dissection of the molecular machinery for targeting and localization of proteins has been studied vigorously. Defined sequence motifs have been identified on proteins which can act as ‘address labels’. A number of sorting signals have been found associated with the cytoplasmic domains of membrane proteins.

### SUMMARY OF THE INVENTION

This invention relates to novel formulations of nucleic acid pharmaceutical products, specifically nucleic acid vaccine products. The nucleic acid products, when introduced directly into muscle cells, induce the production of immune responses which specifically recognize Hepatitis C virus (HCV).

### BRIEF DESCRIPTION OF THE DRAWINGS

FIG. 1 shows the nucleotide sequence of the V1Ra vector.

FIG. 2 is a diagram of the V1Ra vector.

FIG. 3 is a diagram of the Vtpa vector.

FIG. 4 is the VUb vector

FIG. 5 shows an optimized sequence of the HCV core antigen.

FIG. 6 shows V1Ra.HCV1CorePAb, Vtpa.HCV1CorePAb and VUb.HCV1CorePAb.

FIG. 7 shows the Hepatitis C Virus Core Antigen Sequence.

FIG. 8 shows codon utilization in human protein-coding sequences (from Lathe et al.).

FIG. 9 shows an optimized sequence of the HCV E1 protein.

FIG. 10 shows an optimized sequence of the HCV E2 protein.

FIG. 11 shows an optimized sequence of the HCV E1+E2 proteins.

FIG. 12 shows an optimized sequence of the HCV NS5a protein.

FIG. 13 shows an optimized sequence of the HCV NS5b protein.

### DETAILED DESCRIPTION OF THE INVENTION

This invention relates to novel formulations of nucleic acid pharmaceutical products, specifically nucleic acid vaccine products. The nucleic acid vaccine products, when introduced directly into muscle cells, induce the production of immune responses which specifically recognize Hepatitis C virus (HCV).

Non-A, Non-B hepatitis (NANBH) is a transmissible disease (or family of diseases) that is believed to be virally induced, and is distinguishable from other forms of virus-associated liver disease, such as those caused by hepatitis A virus (HAV), hepatitis B virus (HBV), delta hepatitis virus (HDV), cytomegalovirus (CMV) or Epstein-Barr virus (EBV). Epidemiologic evidence suggests that there may be three types of NANBH: the water-borne epidemic type; the blood or needle associated type; and the sporadically occurring (community acquired) type. However, the number of causative agents is unknown. Recently, a new viral species, hepatitis C virus (HCV) has been identified as the primary (if not only) cause of blood-associated NANBH (BB-NANBH). Hepatitis C appears to be the major form of transfusion-associated hepatitis in a number of countries, including the United States and Japan. There is also evidence implicating HCV in induction of hepatocellular carcinoma. Thus, a need exists for an effective method for preventing or treating HCV infection: currently, there is none.

The HCV may be distantly related to the flaviviridae. The Flavivirus family contains a large number of viruses which are small, enveloped pathogens of man. The morphology and composition of Flavivirus particles are known, and are discussed in M. A. Brinton, in “The Viruses: The Togaviridae And Flaviviridae” (Series eds. Fraenkel-Conrat and Wagner, vol. eds. Schlesinger and Schlesinger, Plenum Press, 1996), pp. 327-374. Generally, with respect to morphology, Flaviviruses contain a central nucleocapsid surrounded by a lipid bilayer. Virions are spherical and have a diameter of about 40-50 nm. Their cores are about 25-30 nm in diameter. Along the outer surface of the virion envelope are projections measuring about 5-10 nm in length with terminal knobs about 2 nm in diameter. Typical examples of the family include Yellow Fever virus, West Nile virus, and Dengue Fever virus. They possess positive-stranded RNA genomes (about 11,000 nucleotides) that are slightly larger than that of HCV and encode a polyprotein precursor of about 3500 amino acids. Individual viral proteins are cleaved from this precursor polypeptide.

The genome of HCV appears to be single-stranded RNA containing about 10,000 nucleotides. The genome is positive-stranded, and possesses a continuous translational open reading frame (ORF) that encodes a polyprotein of about 3,000 amino acids. In the ORF, the structural proteins appear to be encoded in approximately the first quarter of the N-terminal region, with the majority of the polyprotein attributed to non-structural proteins. When compared with all known viral sequences, small but significant co-linear homologies are observed with the nonstructural proteins of the Flavivirus family, and with the pestiviruses (which are now also considered to be part of the Flavivirus family).

Intramuscular inoculation of polynucleotide constructs, i.e., DNA plasmids encoding proteins have been shown to result in the generation of the encoded protein in situ in muscle cells. By using cDNA plasmids encoding viral proteins, both antibody and CTL responses were generated, providing homologous and heterologous protection against subsequent challenge with either the homologous or cross-strain protection, respectively. Each of these types of immune responses offers a potential advantage over existing vaccination strategies. The use of PNVs (polynucleotide vaccines) to generate antibodies may result in an increased duration of the antibody responses as well as the provision of an antigen that can have both the exact sequence of the clinically circulating strain of virus as well as the proper post-translational modifications and conformation of the native protein (vs. a recombinant protein). The generation of CTL responses by this means offers the benefits of cross-strain protection without the use of a live potentially pathogenic vector or attenuated virus.

The standard techniques of molecular biology for preparing and purifying DNA constructs enable the preparation of the DNA therapeutics of this invention. While standard techniques of molecular biology are therefore sufficient for the production of the products of this invention, the specific constructs disclosed herein provide novel therapeutics which surprisingly produce cross-strain protection, a result heretofore unattainable with standard inactivated whole virus or subunit protein vaccines.

The amount of expressible DNA to be introduced to a vaccine recipient will depend on the strength of the transcriptional and translational promoters used in the DNA construct, and on the immunogenicity of the expressed gene product. In general, an immunologically or prophylactically effective dose of about 1 μg to 1 mg, and preferably about 10 μg to 300 μg is administered directly into muscle tissue. Subcutaneous injection, intradermal introduction, impression through the skin, and other modes of administration such as intraperitoneal, intravenous, or inhalation delivery are also contemplated. It is also contemplated that booster vaccinations are to be provided.

The DNA may be naked, that is, unassociated with any proteins, adjuvants or other agents which impact on the recipients immune system. In this case, it is desirable for the DNA to be in a physiologically acceptable solution, such as, but not limited to, sterile saline or sterile buffered saline. Alternatively, the DNA may be associated with surfactants, liposomes, such as lecithin liposomes or other liposomes known in the art, as a DNA-liposome mixture, (see for example WO93/24640) or the DNA may be associated with an adjuvant known in the art to boost immune responses, such as a protein or other carrier. Agents which assist in the cellular uptake of DNA, such as, but not limited to, calcium ions, detergents, viral proteins and other transfection facilitating agents may also be used to advantage. These agents are generally referred to as transfection facilitating agents and as pharmaceutically acceptable carriers. As used herein, the term gene refers to a segment of nucleic acid which encodes a discrete polypeptide. The term pharmaceutical, and vaccine are used interchangeably to indicate compositions useful for inducing immune responses. The terms construct, and plasmid are used interchangeably. The term vector is used to indicate a DNA into which genes may be cloned for use according to the method of this invention.

The following examples are provided to further define the invention, without limiting the invention to the specifics of the examples.

### EXAMPLE 1

V1J Expression Vectors:

V1J is derived from vectors V1 and pUC18, a commercially available plasmid. V1 was digested with SspI and EcoRI restriction enzymes producing two fragments of DNA. The smaller of these fragments, containing the CMVintA promoter and Bovine Growth Hormone (BGH) transcription termination elements which control the expression of heterologous genes, was purified from an agarose electrophoresis gel. The ends of this DNA fragment were then “blunted” using the T4 DNA polymerase enzyme in order to facilitate its ligation to another “blunt-ended” DNA fragment.

pUC18 was chosen to provide the “backbone” of the expression vector. It is known to produce high yields of plasmid, is well-characterized by sequence and function, and is of minimum size. We removed the entire lac operon from this vector, which was unnecessary for our purposes and may be detrimental to plasmid yields and heterologous gene expression, by partial digestion with the HaeII restriction enzyme. The remaining plasmid was purified from an agarose electrophoresis gel, blunt-ended with the T4 DNA polymerase, treated with calf intestinal alkaline phosphatase, and ligated to the CMVintA/BGH element described above. Plasmids exhibiting either of two possible orientations of the promoter elements within the pUC backbone were obtained. One of these plasmids gave much higher yields of DNA in E. coli and was designated V1J. This vector's structure was verified by sequence analysis of the junction regions and was subsequently demonstrated to give comparable or higher expression of heterologous genes compared with V1. The ampicillin resistance marker was replaced with the neomycin resistance marker to yield vector V1Jneo.

An Sfi I site was added to V1Jneo to facilitate integration studies. A commercially available 13 base pair Sfi I linker (New England BioLabs) was added at the Kpn I site within the BGH sequence of the vector. V1Jneo was linearized with Kpn I, gel purified, blunted by T4 DNA polymerase, and ligated to the blunt Sfi I linker. Clonal isolates were chosen by restriction mapping and verified by sequencing through the linker. The new vector was designated V1Jns. Expression of heterologous genes in V1Jns (with Sfi I) was comparable to expression of the same genes in V1Jneo (with Kpn 1).

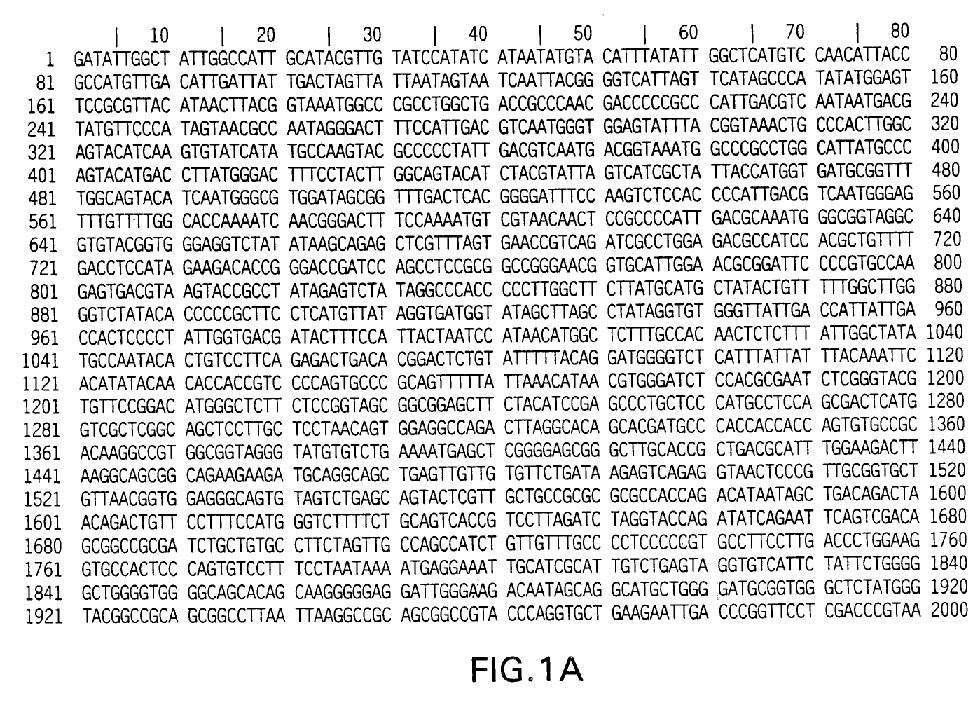

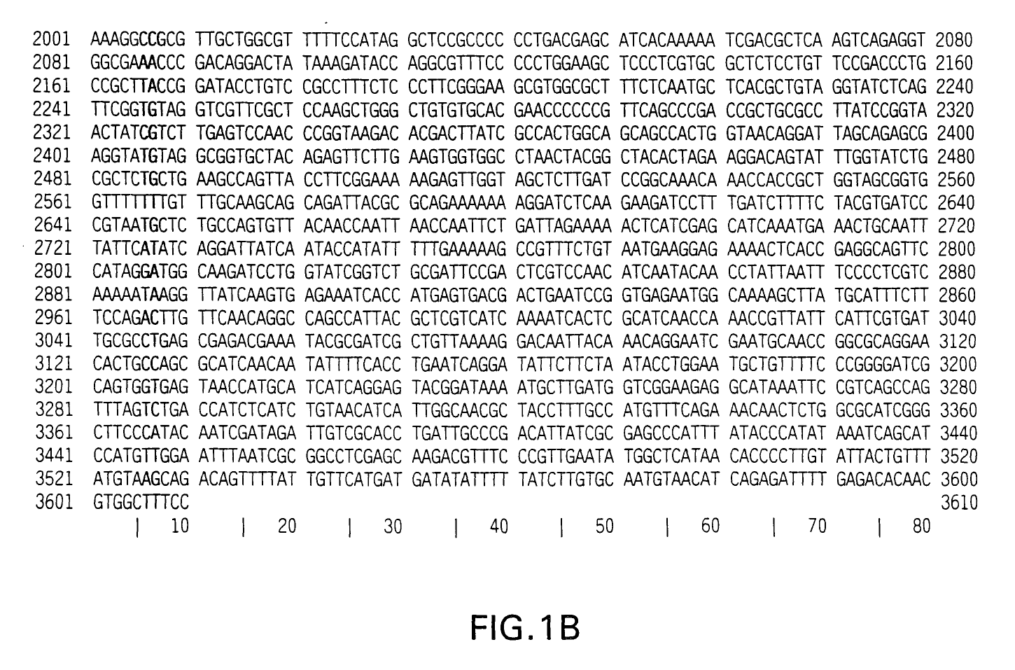

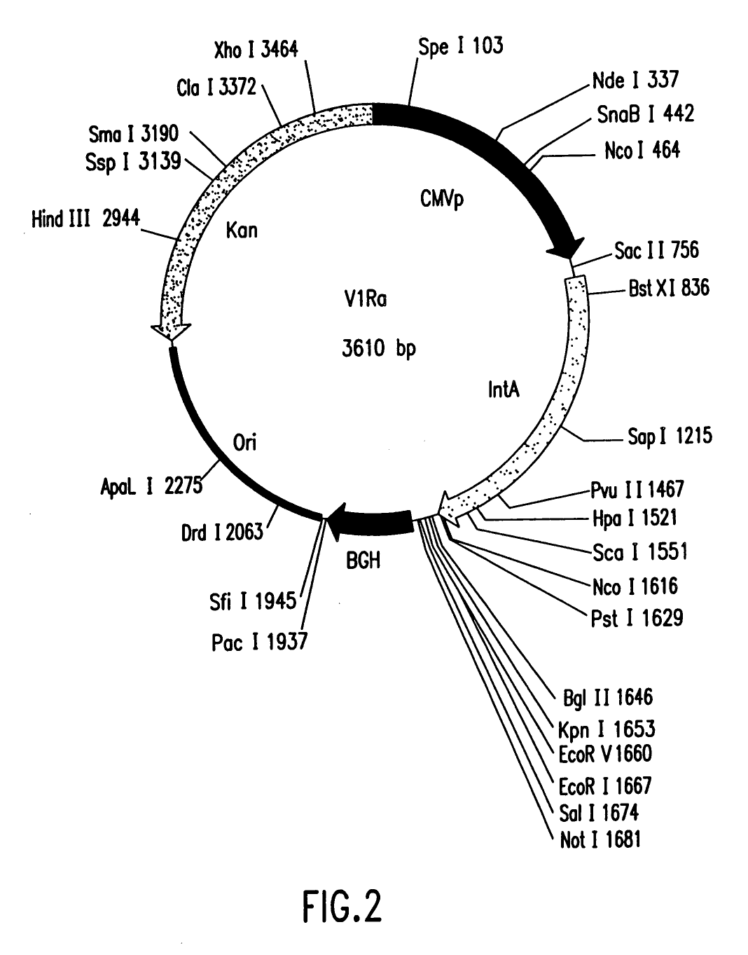

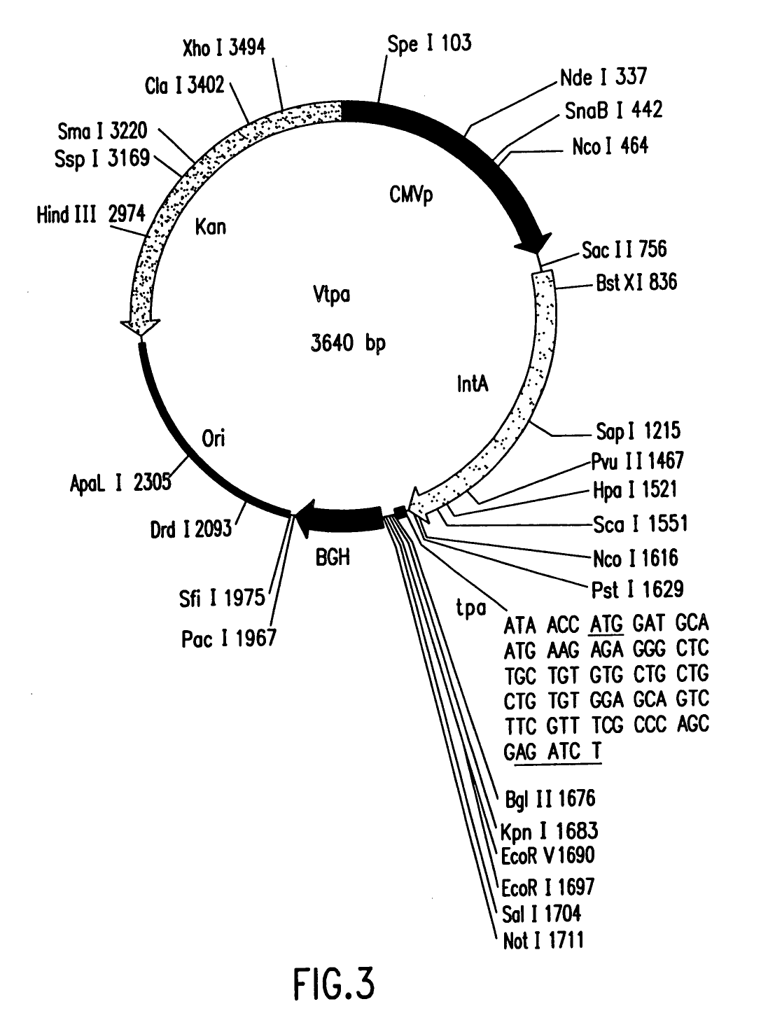

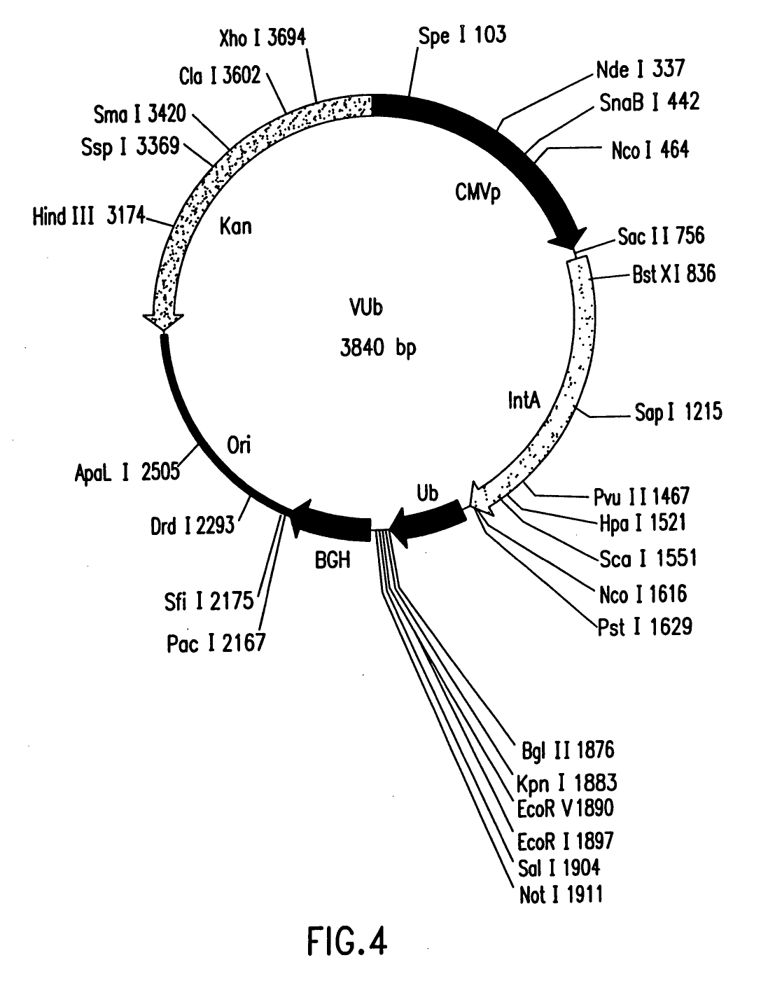

Vector V1Ra (Sequence is shown in FIG. 1; map is shown in FIG. 2) was derived from vector V1R, a derivative of the V1Jns vector. Multiple cloning sites (BglII, KpnI, EcoRV, EcoRI, SalI, and NotI) were introduced into V1R to create the V1 Ra vector to improve the convenience of subcloning. V1Ra vector derivatives containing the tpa leader sequence and ubiquitin sequence were generated (Vtpa (FIG. 3) and Vub (FIG. 4), respectively). Expression of viral antigen from Vtpa vector will target the antigen protein into the exocytic pathway, thus producing a secretable form of the antigen proteins. These secreted proteins are likely to be captured by professional antigen presenting cells, such as macrophages and dendritic cells, and processed and presented by class II molecules to activate CD4+ Th cells. They also are more likely to efficiently simulate antibody responses. Expression of viral antigen through VUb vector will produce a ubiquitin and antigen fusion protein. The uncleavable ubiquitin segment (glycine to alanine change at the cleavage site, Butt et al., JBC 263:16364, 1988) will target the viral antigen to ubiquitin-associated proteasomes for rapid degradation. The resulting peptide fragments will be transported into the ER for antigen presentation by class I molecules. This modification is attempted to enhance the class I molecule-restricted CTL responses against the viral antigen (Townsend et al, JEM 168:1211, 1988).

### EXAMPLE 2

Design and Construction of the Synthetic Genes

A. Design of Synthetic Gene Segments for HCV Gene Expression:

Gene segments were converted to sequences having identical translated sequences (except where noted) but with alternative codon usage as defined by R. Lathe in a research article from J. Molec. Biol. Vol. 183, pp. 1-12 (1985) entitled “Synthetic Oligonucleotide Probes Deduced from Amino Acid Sequence Data: Theoretical and Practical Considerations”. The methodology described below was based on our hypothesis that the known inability to express a gene efficiently in mammalian cells is a consequence of the overall transcript composition. Thus, using alternative codons encoding the same protein sequence may remove the constraints on HCV gene expression. Inspection of the codon usage within HCV genome revealed that a high percentage of codons were among those infrequently used by highly expressed human genes. The specific codon replacement method employed may be described as follows employing data from Lathe et al.:

1. Identify placement of codons for proper open reading frame.

2. Compare wild type codon for observed frequency of use by human genes (refer to Table 3 in Lathe et al.).

3. If codon is not the most commonly employed, replace it with an optimal codon for high expression based on data in Table 5.

4. Inspect the third nucleotide of the new codon and the first nucleotide of the adjacent codon immediately 3′- of the first. If a 5′-CG-3′ pairing has been created by the new codon selection, replace it with the choice indicated in Table 5.

5. Repeat this procedure until the entire gene segment has been replaced.

6. Inspect new gene sequence for undesired sequences generated by these codon replacements (e.g., “ATTTA” sequences, inadvertent creation of intron splice recognition sites, unwanted restriction enzyme sites, etc.) and substitute codons that eliminate these sequences.

7. Assemble synthetic gene segments and test for improved expression.

B. HCV Core Antigen Sequence

The consensus core sequence of HCV was adopted from a generalized core sequence reported by Bukh et al. (PNAS, 91:8239, 1994). This core sequence contains all the identified CTL epitopes in both human and mouse. The gene is composed of 573 nucleotides and encodes 191 amino acids. The predicted molecular weight is about 23 kDa.

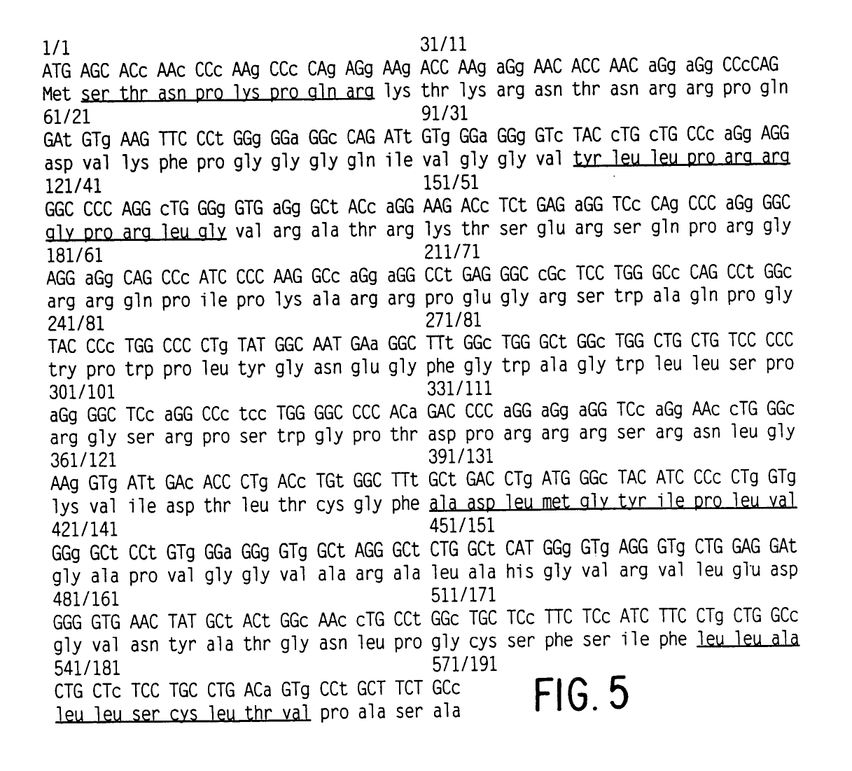

The codon replacement was conducted to eliminate codons which may hinder the expression of the HCV core protein in transfected mammalian cells in order to maximize the translational efficiency of DNA vaccine. Twenty three point two percent (23.2%) of nucleotide sequence (133 out of 573 nucleotides) were altered, resulting in changes of 61.3% of the codons (117 out 191 codons) in the core antigen sequence. The optimized nucleotide sequence of HCV core is shown in FIG. 5.

C. Construction of the Synthetic Core Gene

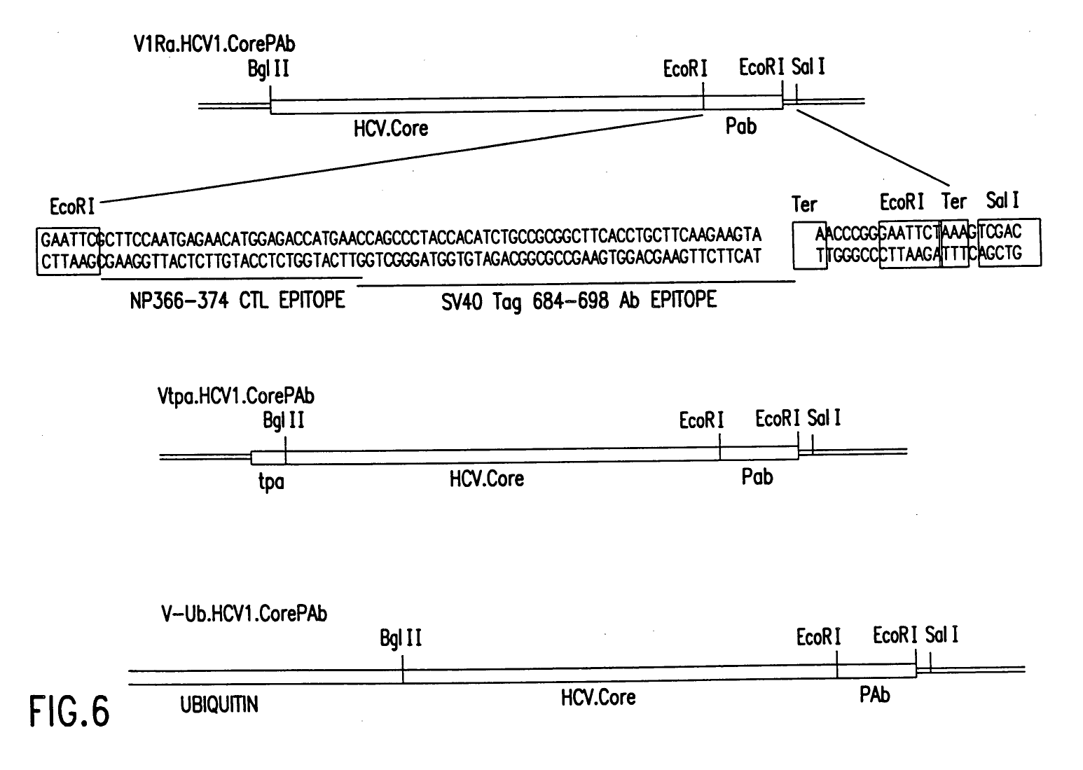

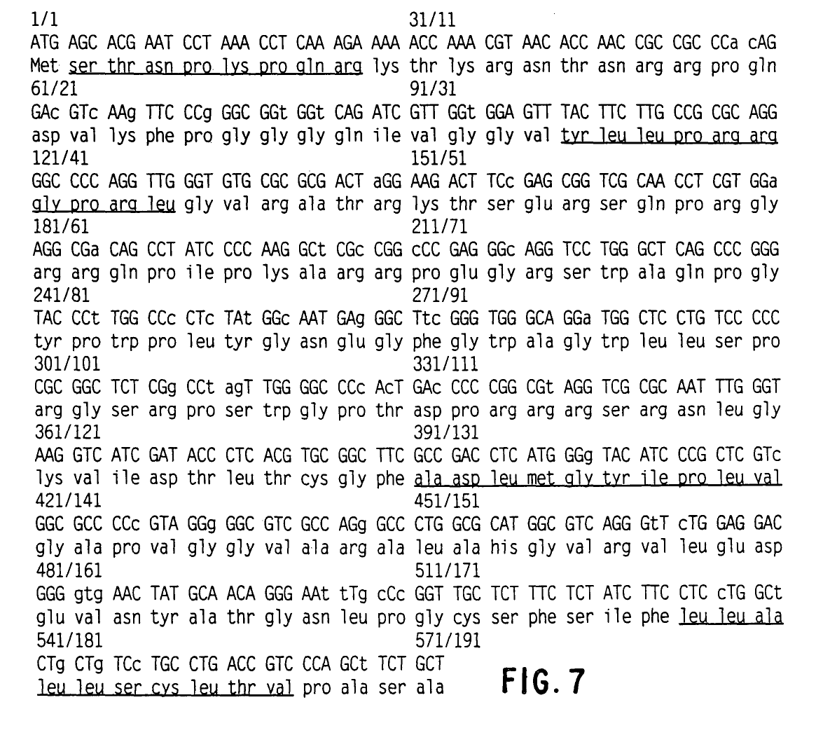

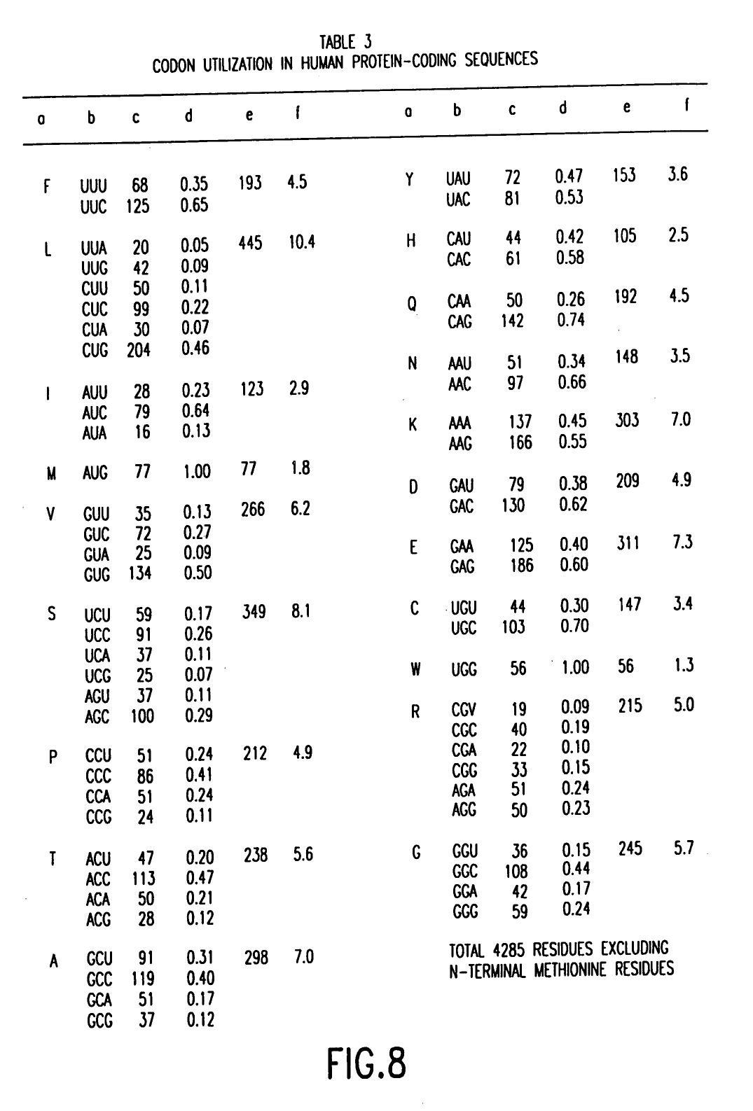

The optimized HCV core gene (FIG. 5) was constructed as a synthetic gene annealed from multiple synthetic oligonucleotides. To facilitate the identification and evaluation of the synthetic gene expression in cell culture and its immunogenicity in mice, a CTL epitope derived from influenza virus nucleoprotein residues 366-374 and an antibody epitope sequence derived from SV40 T antigen residues 684-698 were tagged to the carboxyl terminal of the core sequence (FIG. 6). For clinical use it may be desired to express the core sequence without the nucleoprotein 366-374 and SV40 T 684-698 sequences. For this reason, the sequence of the two epitopes is flanked by two EcoRI sites which will be used to excise this fragment of sequence at a later time. Thus an embodiment of the invention for clinical use could consist of the V1Ra.HCV1CorePAb, Vtpa.HCV1CorePAb, or VUb.HCV1CorePAb plasmids that had been cut with EcoRI, annealed, and ligated to yield plasmids V1Ra.HCV 1 Core, Vtpa.HCV1 Core, and VUb.HCV1 Core.

The synthetic gene was built as three separate segments in three vectors, nucleotides 1 to 80 in V1Ra, nucleotides 80 to 347 (BstXI site) in pUC1S, and nucleotides 347 to 573 plus the two epitope sequence in pUC18. All the segments were verified by DNA sequencing, and joined together in V1Ra vector.

D. HCV Gene Expression Constructs:

In each case, the junction sequences from the 5′ promoter region (CMVintA) into the cloned gene is shown. The position at which the junction occurs is demarcated by a “/”, which does not represent any discontinuity in the sequence.

The nomenclature for these constructs follows the convention: “Vector name-HCV strain-gene”.

```
                          V1Ra.HCV1.CorePAb
---IntA--AGA TCT ACC / ATG AGC--HCV.Core.--GCC / GAA TTC GCT TCC--
PAb Sequence--TAA / ACC CGG GAA TTC TAA A / GTC GAC--BGH---
                          Vtpa.HCV1.CorePAb
---IntA--ATC ACC / ATG GAT--tpa leader--GAG ATC-TTC / ATG AGC--
HCV.Core.--GCC / GAA TTC GCT TCC--PAb Sequence--TAA / ACC CGG GAA
TTC TAA A / GTC GAC--BGH---
                          VUb.HCV1.CorePAb.
---IntA--AGA TCC ACC / ATG CAG--Ubiquitin--GGT GCA GAT CTG/ ATG AGC--
HCV.Core.--GCC / GAA TTC GCT TCC--PAb Sequence--TAA / ACC CGG GAA
TTC TAA A / GTC GAC--BGH--
                          V1Ra.HCV1.Core
---IntA--AGA TCT ACC / ATG AGC--HCV.Core.--GCC / TAA A / GTC GAC--
BGH---
                          Vtpa.HCV1.Core
---IntA--ATC ACC / ATG GAT--tpa leader--GAG ATC-TTC / ATG AGC--
HCV.Core.--GCC / TAA A / GTC GAC--BGH---
                          VUb.HCV1.Core
---IntA--AGA TCC ACC / ATG CAG--Ubiquitin--GGT GCA GAT CTG/ ATG AGC--
HCV.Core.--GCC / TAA A / GTC GAC--BGH--
```

E. Other Synthetic HCV Genes

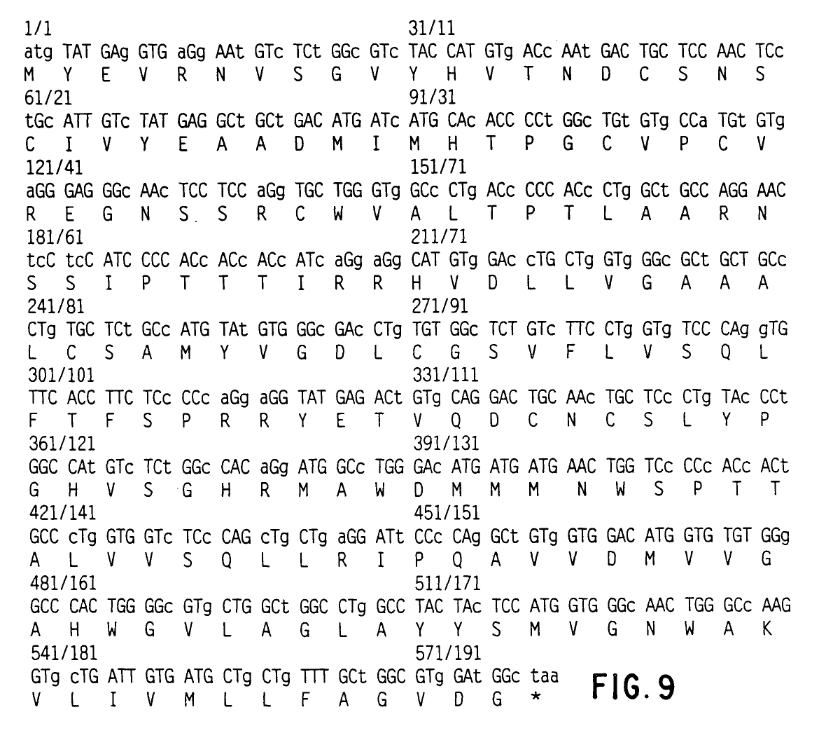

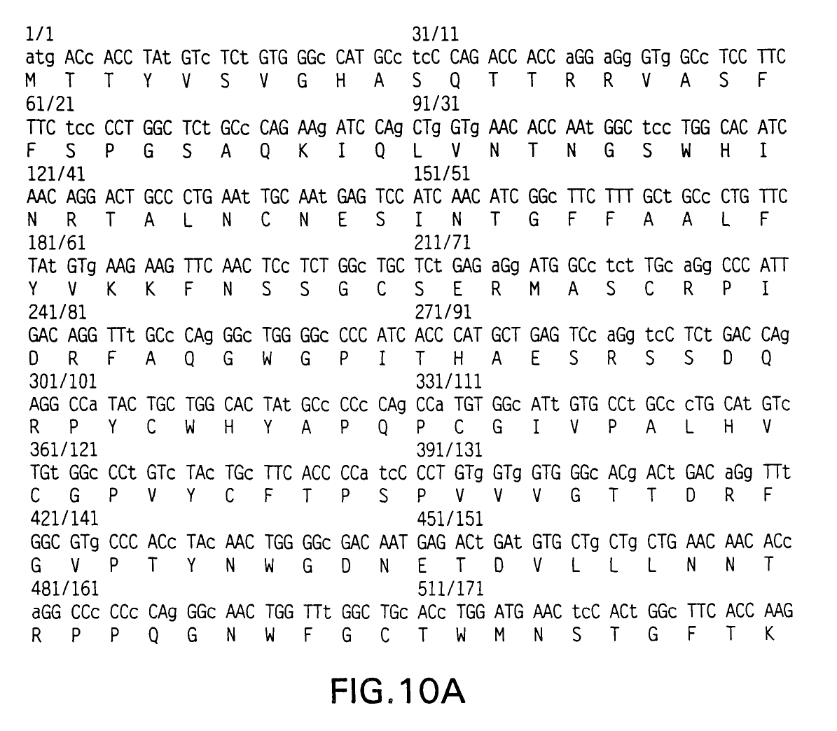

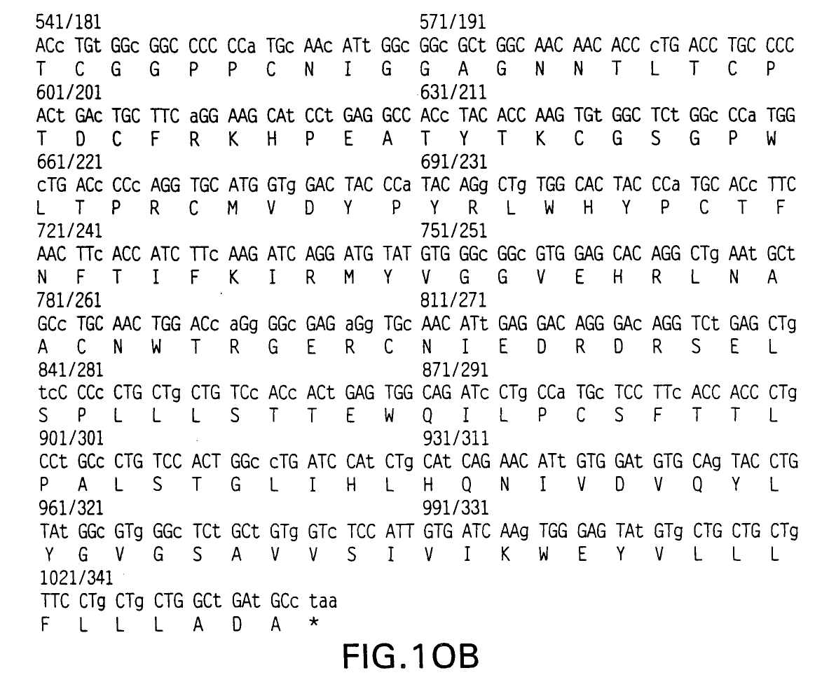

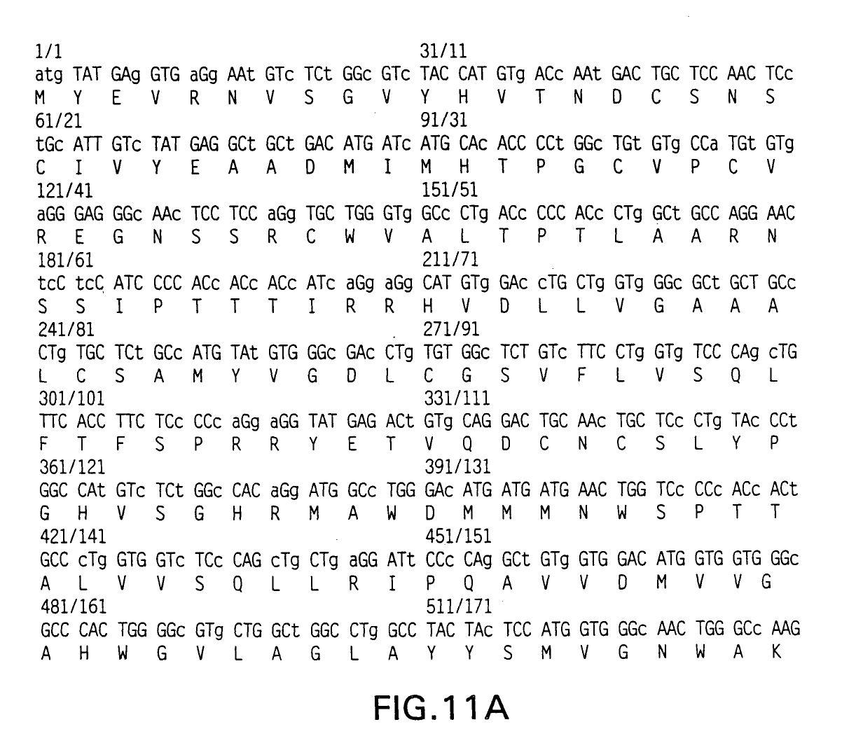

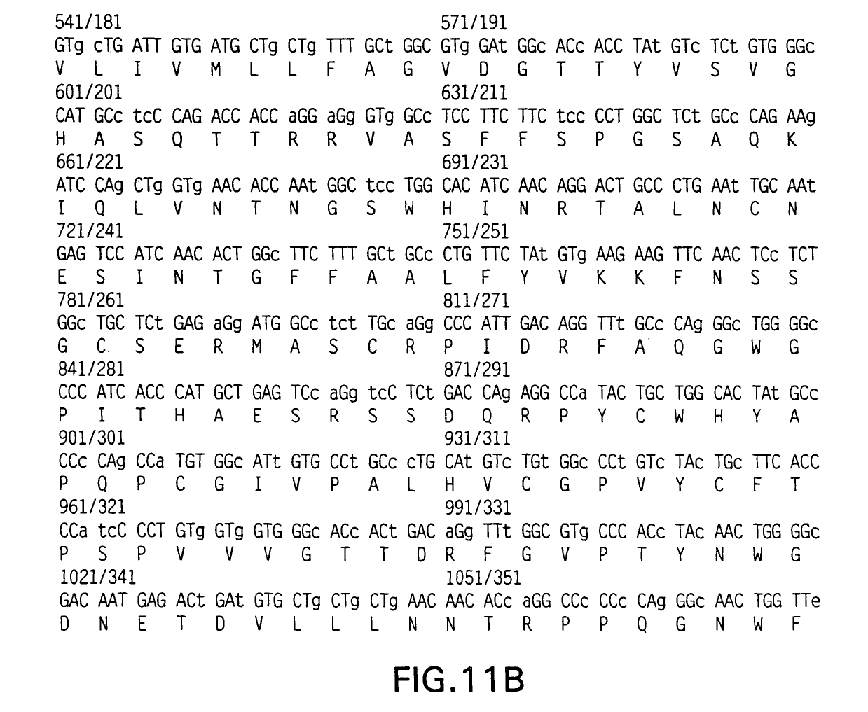

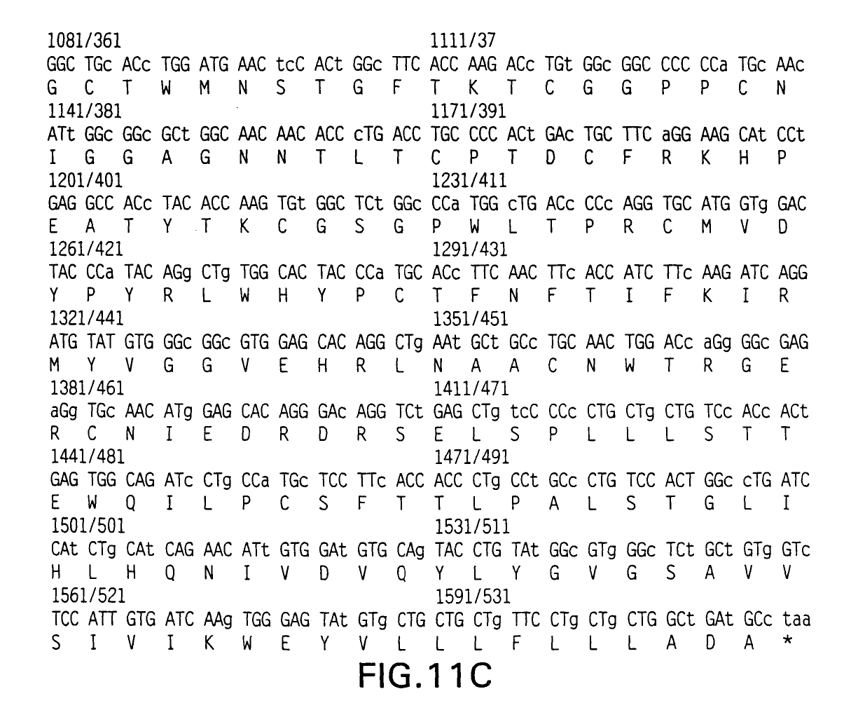

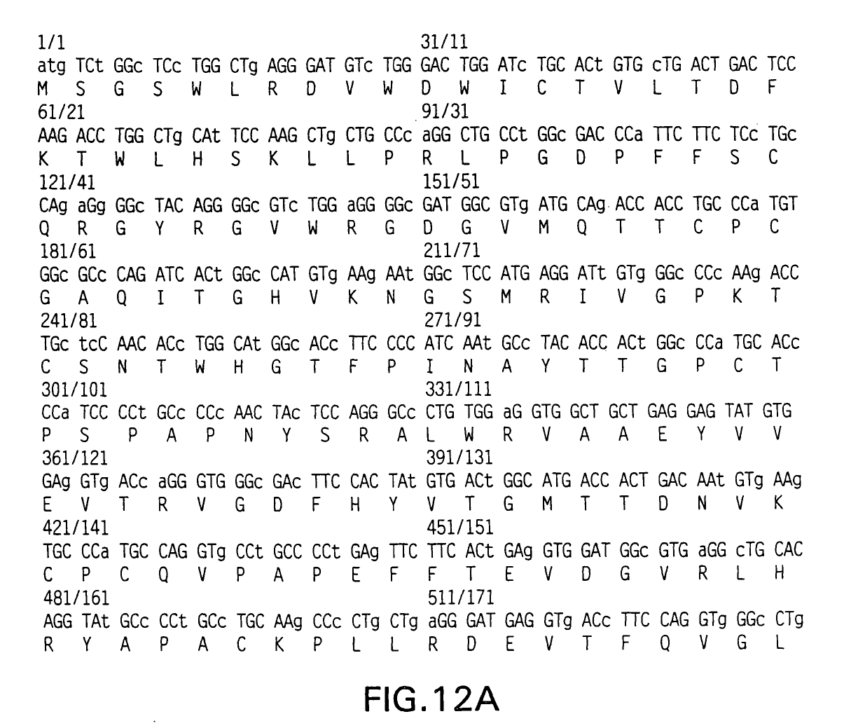

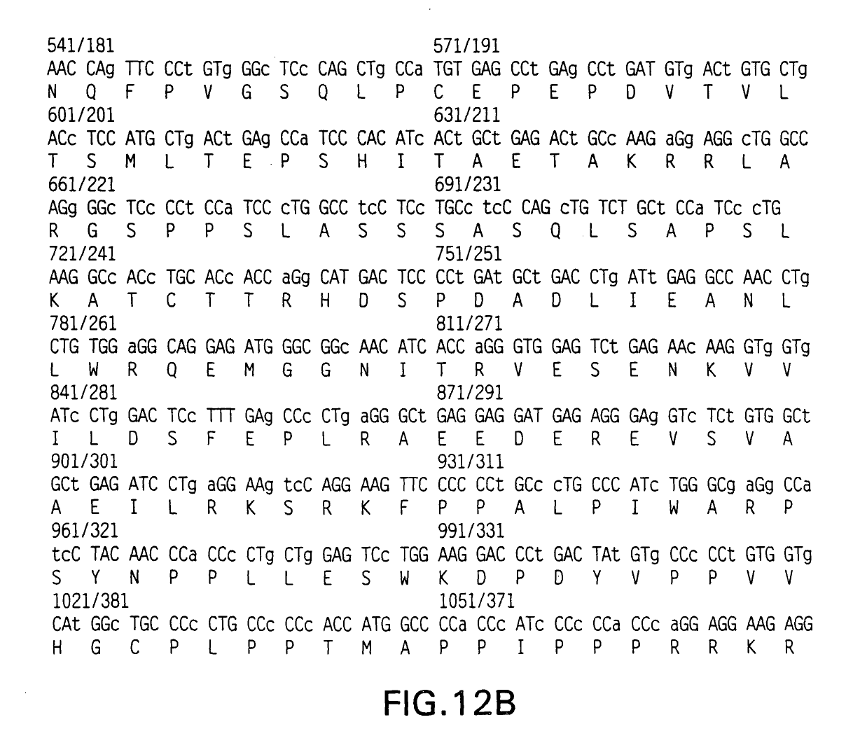

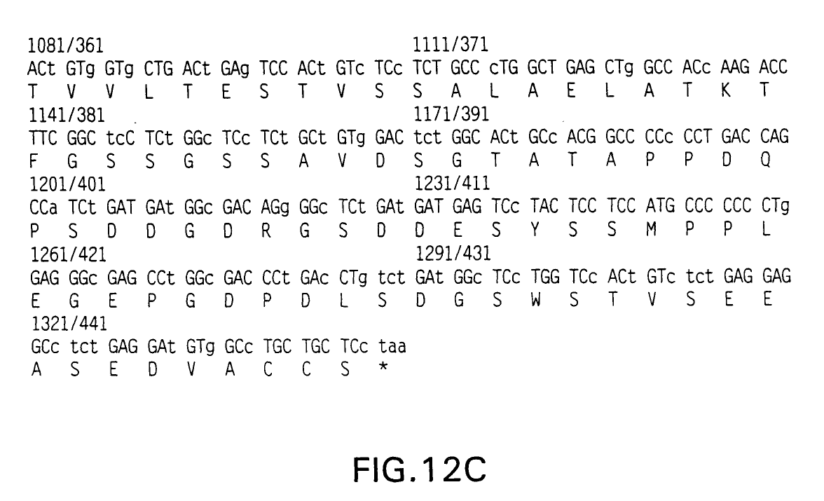

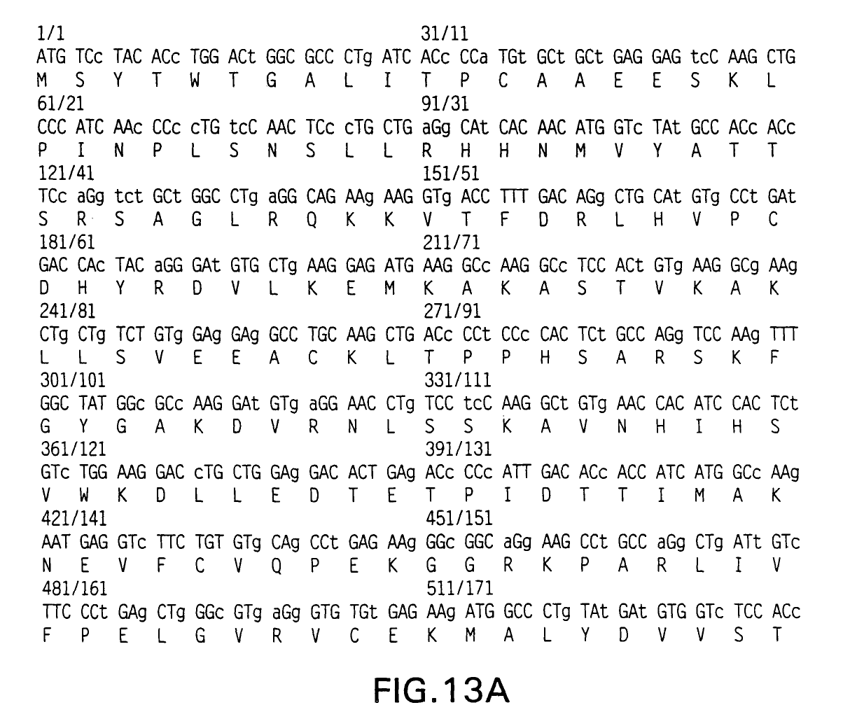

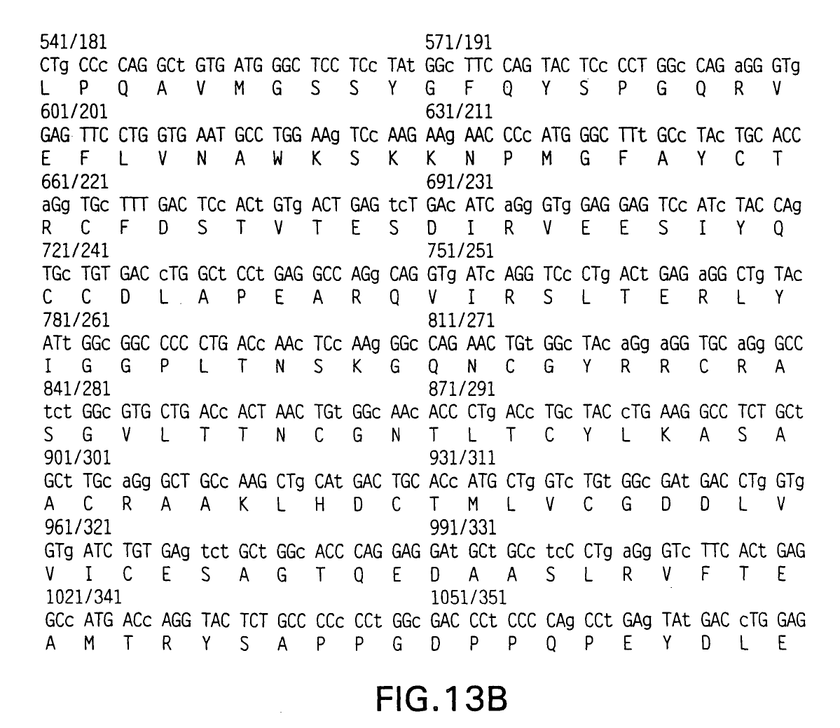

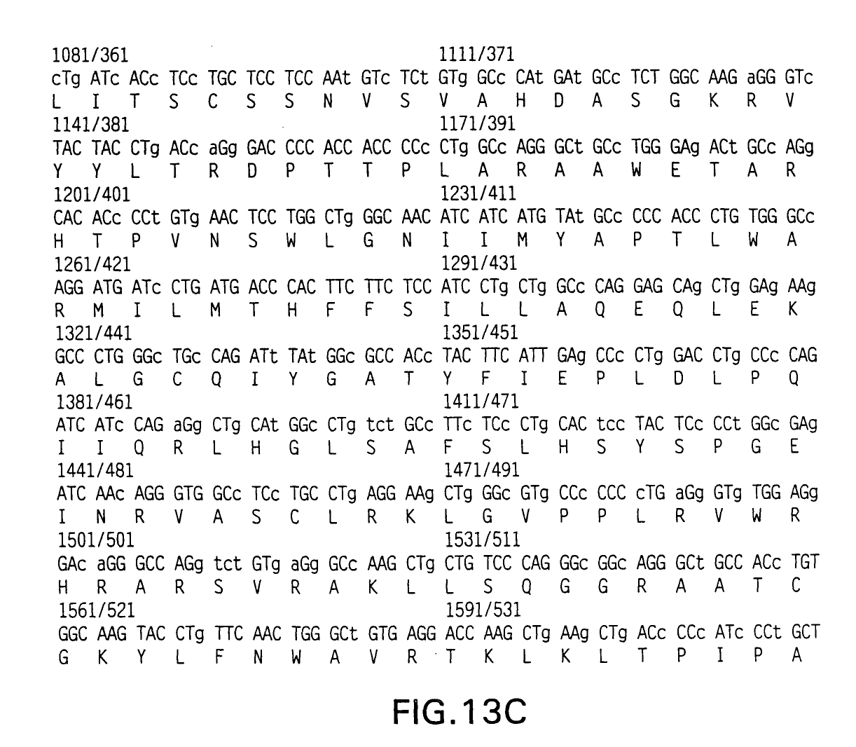

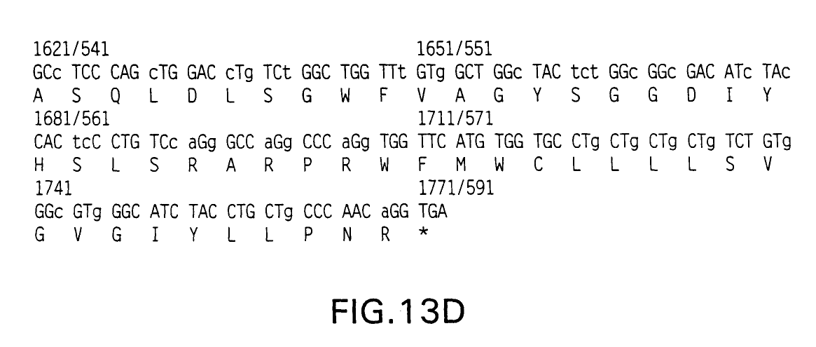

Using similar codon optimization techniques, synthetic genes encoding the HCV E1 (FIG. 9), HCV E2 (FIG. 10), HCV E1+E2 (FIG. 11), HCV NS5a (FIG. 12) and HCV NS5b (FIG. 13) proteins were created.

1. A synthetic polynucleotide comprising a DNA sequence encoding an HCV protein selected from the group consisting of HCV core protein, HCV E1 protein, HCV E1+E2 protein, HCV NS5a protein, HCV NS5b protein and fragments thereof, the DNA sequence comprising codons optimized for expression in a vertebrate host.

2. A plasmid vector comprising the polynucleotide of claim 1, the plasmid vector being suitable for immunization of a vertebrate host.

3. The polynucleotide of claim 1 which is HCV genotype I/Ia core.

4-7. (canceled)

8. A method for inducing immune responses in a vertebrate against HCV epitopes which comprises introducing between 1 ng and 100 mg of the polynucleotide of claim 1 into the tissue of the vertebrate.

9. A method for inducing immune responses against infection or disease caused by HCV which comprises introducing into the tissue of a vertebrate the polynucleotide of claim 1.

10. A vaccine for inducing immune responses against HCV infection which comprises the polynucleotide of claim 1 and a pharmaceutically acceptable carrier.

11. A method for inducing anti-HCV immune responses in a primate which comprises introducing the polynucleotide of claim 1 into the tissue of said primate and concurrently administering interleukin-12 parenterally.

12. A method of inducing an antigen presenting cell to stimulate cytotoxic and helper T-cell proliferation an effector functions including lymphokine secretion specific to HCV antigens which comprises exposing cells of a vertebrate in vivo to the polynucleotide of claim 1.

13. A method of treating a patient in need of such treatment comprising administering to the patient the polynucleotide of claim 1 in combination with interferon-alpha, Ribavirin, Zidovudine, or other pharmaceutically acceptable antiviral agents.

14. A pharmaceutical composition comprising the polynucleotide of claim 1.

15. A method of inducing an immune response comprising administering the polynucleotide of claim 1 to a patient, the administration of the polynucleotide antedating or coinciding or following administration to the patient of a subunit, recombinant, recombinant live vector, inactivated, recombinant inactivated vector, or live attenuated HCV vaccine.

16. A method for inducing immune responses in a vertebrate against HCV epitopes which comprises introducing between 1 ng and 100 mg of the polynucleotide of claim 2 into the tissue of the vertebrate.

17. A method for inducing immune responses against infection or disease caused by HCV which comprises introducing into the tissue of a vertebrate the polynucleotide of claim 2.

18. A vaccine for inducing immune responses against HCV infection which comprises the polynucleotide of claim 2 and a pharmaceutically acceptable carrier.

19. A method for inducing anti-HCV immune responses in a primate which comprises introducing the polynucleotide of claim 2 into the tissue of said primate and concurrently administering interleukin 12 parenterally.

20. A method of inducing an antigen presenting cell to stimulate cytotoxic and helper T-cell proliferation an effector functions including lymphokine secretion specific to HCV antigens which comprises exposing cells of a vertebrate in vivo to the polynucleotide of claim 2.

21. A method of treating a patient in need of such treatment comprising administering to the patient the polynucleotide of claim 2 in combination with interferon-alpha, Ribavirin, Zidovudine, or other pharmaceutically acceptable antiviral agents.

22. A pharmaceutical composition comprising the polynucleotide of claim 2.

23. A method of inducing an immune response comprising administering the polynucleotide of claim 2 to a patient, the administration of the polynucleotide antedating or coinciding or following administration to the patient of a subunit, recombinant, recombinant live vector, inactivated, recombinant inactivated vector, or live attenuated HCV vaccine.

24-25. (canceled)

26. The DNA sequence of claim 1 selected from the group consisting of a nucleotide sequence shown in FIG. 5, FIG. 9, FIG. 10, FIG. 11, FIG. 12 and FIG.

13.


SEQUENCE LISTING (25 sequences)

SEQ ID NO: 1 - DNA, 3610 bp, Artificial Sequence
  Modified Vector Sequence
gatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctaggtaccagatatcagaattcagtcgacagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacggccgcagcggccttaattaaggccgcagcggccgtacccaggtgctgaagaattgacccggttcctcgacccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtgatcccgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcc

SEQ ID NO: 2 - DNA, 573 bp, Artificial Sequence
  Optimized sequence encoding HCV core antigen
atgagcaccaaccccaagccccagaggaagaccaagaggaacaccaacaggaggccccaggatgtgaagttccctgggggaggccagattgtgggaggggtctacctgctgcccaggaggggccccaggctgggggtgagggctaccaggaagacctctgagaggtcccagcccaggggcaggaggcagcccatccccaaggccaggaggcctgagggccgctcctgggcccagcctggctacccctggcccctgtatggcaatgaaggctttggctgggctggctggctgctgtcccccaggggctccaggccctcctggggccccacagaccccaggaggaggtccaggaacctgggcaaggtgattgacaccctgacctgtggctttgctgacctgatgggctacatccccctggtgggggctcctgtgggaggggtggctagggctctggctcatggggtgagggtgctggaggatggggtgaactatgctactggcaacctgcctggctgctccttctccatcttcctgctggccctgctctcctgcctgacagtgcctgcttctgcc

SEQ ID NO: 3 - protein, 191 aa, Hepatitis C Virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGFGWAGWLLSPRGSRPSWGPTDPRRRSRNLGKVIDTLTCGFADLMGYIPLVGAPVGGVARALAHGVRVLEDGVNYATGNLPGCSFSIFLLALLSCLTVPASA

SEQ ID NO: 4 - DNA, 103 bp, Artificial Sequence
  Modified Vector Sequence
gaattcgcttccaatgagaacatggagaccatgaaccagccctaccacatctgccgcggcttcacctgcttcaagaagtaaacccgggaattctaaagtcgac

SEQ ID NO: 5 - DNA, 573 bp, Hepatitis C Virus
atgagcacgaatcctaaacctcaaagaaaaaccaaacgtaacaccaaccgccgcccacaggacgtcaagttcccgggcggtggtcagatcgttggtggagtttacttgttgccgcgcaggggccccaggttgggtgtgcgcgcgactaggaagacttccgagcggtcgcaacctcgtggaaggcgacagcctatccccaaggctcgccggcccgagggcaggtcctgggctcagcccgggtacccttggcccctctatggcaatgagggcttcgggtgggcaggatggctcctgtccccccgcggctctcggcctagttggggccccactgacccccggcgtaggtcgcgcaatttgggtaaggtcatcgataccctcacgtgcggcttcgccgacctcatggggtacatcccgctcgtcggcgcccccgtagggggcgtcgccagggccctggcgcatggcgtcagggttctggaggacggggtgaactatgcaacagggaatttgcccggttgctctttctctatcttcctcctggctctgctgtcctgcctgaccgtcccagcttctgct

SEQ ID NO: 6 - DNA, 582 bp, Artificial Sequence
  Optimized sequence encoding HCV E1 protein
atgtatgaggtgaggaatgtctctggcgtctaccatgtgaccaatgactgctccaactcctgcattgtctatgaggctgctgacatgatcatgcacacccctggctgtgtgccatgtgtgagggagggcaactcctccaggtgctgggtggccctgacccccaccctggctgccaggaactcctccatccccaccaccaccatcaggaggcatgtggacctgctggtgggcgctgctgccctgtgctctgccatgtatgtgggcgacctgtgtggctctgtcttcctggtgtcccagctgttcaccttctcccccaggaggtatgagactgtgcaggactgcaactgctccctgtaccctggccatgtctctggccacaggatggcctgggacatgatgatgaactggtcccccaccactgccctggtggtctcccagctgctgaggatcccccaggctgtggtggacatggtggtgggcgcccactggggcgtgctggctggcctggcctactactccatggtgggcaactgggccaaggtgctgattgtgatgctgctgtttgctggcgtggatggctaa

SEQ ID NO: 7 - protein, 193 aa, Hepatitis C Virus
MYEVRNVSGVYHVTNDCSNSCIVYEAADMIMHTPGCVPCVREGNSSRCWVALTPTLAARNSSIPTTTIRRHVDLLVGAAALCSAMYVGDLCGSVFLVSQLFTFSPRRYETVQDCNCSLYPGHVSGHRMAWDMMMNWSPTTALVVSQLLRIPQAVVDMVVGAHWGVLAGLAYYSMVGNWAKVLIVMLLFAGVDG

SEQ ID NO: 8 - DNA, 1044 bp, Artificial Sequence
  Optimized sequence encoding HCV E2 protein
atgaccacctatgtctctgtgggccatgcctcccagaccaccaggagggtggcctccttcttctcccctggctctgcccagaagatccagctggtgaacaccaatggctcctggcacatcaacaggactgccctgaattgcaacgagtccatcaacactggcttctttgctgccctgttctatgtgaagaagttcaactcctctggctgctctgagaggatggcctcctgcaggcccattgacaggtttgcccagggctggggccccatcacccatgctgagtccaggtcctctgaccagaggccatactgctggcactatgccccccagccatgtggcattgtgcctgccctgcaggtctgtggccctgtctactgcttcaccccatcccctgtggtggtgggcaccactgacaggtttggcgtgcccacctacaactggggcgacaatgagactgatgtgctgctgctgaacaacaccaggcccccccagggcaactggtttggctgcacctggatgaactccactggcttcaccaagacctgtggcggccccccatgcaacattggcggcgctggcaacaacaccctgacctgccccactgactgcttcaggaagcatcctgaggccacctacaccaagtgtggctctggcccatggctgacccccaggtgcatggtggactacccatacaggctgtggcactacccatgcaccttcaacttcaccatcttcaagatcaggatgtatgtgggcggcgtggagcacaggctgaatgctgcctgcaactggaccaggggcgagaggtgcaacattgaggacagggacaggtctgagctgtcccccctgctgctgtccaccactgagtggcagatcctgccatgctccttcaccaccctgcctgccctgtccactggcctgatccatctgcatcagaacattgtggatgtgcagtacctgtacggcgtgggctccgctgtggtctccattgtgatcaagtgggagtatgtgctgctgctgttcctgctgctggctgatgcctaa

SEQ ID NO: 9 - protein, 347 aa, Hepatitis C Virus
MTTYVSVGHASQTTRRVASFFSPGSAQKIQLVNTNGSWHINRTALNCNESINTGFFAALFYVKKFNSSGCSERMASCRPIDRFAQGWGPITHAESRSSDQRPYCWHYAPQPCGIVPALQVCGPVYCFTPSPVVVGTTDRFGVPTYNWGDNETDVLLLNNTRPPQGNWFGCTWMNSTGFTKTCGGPPCNIGGAGNNTLTCPTDCFRKHPEATYTKCGSGPWLTPRCMVDYPYRLWHYPCTFNFTIFKIRMYVGGVEHRLNAACNWTRGERCNIEDRDRSELSPLLLSTTEWQILPCSFTTLPALSTGLIHLHQNIVDVQYLYGVGSAVVSIVIKWEYVLLLFLLLADA

SEQ ID NO: 10 - DNA, 1620 bp, Artificial Sequence
  Optimized sequence encoding HCV E1 + E2 proteins
atgtatgaggtgaggaatgtctctggcgtctaccatgtgaccaatgactgctccaactcctgcattgtctatgaggctgctgacatgatcatgcacacccctggctgtgtgccatgtgtgagggagggcaactcctccaggtgctgggtggccctgacccccaccctggctgccaggaactcctccatccccaccaccaccatcaggaggcatgtggacctgctggtgggcgctgctgccctgtgctctgccatgtatgtgggcgacctgtgtggctctgtcttcctggtgtcccagctgttcaccttctcccccaggaggtatgagactgtgcaggactgcaactgctccctgtaccctggccatgtctctggccacaggatggcctgggacatgatgatgaactggtcccccaccactgccctggtggtctcccagctgctgaggatcccccaggctgtggtggacatggtggtgggcgcccactggggcgtgctggctggcctggcctactactccatggtgggcaactgggccaaggtgctgattgtgatgctgctgtttgctggcgtggatggcaccacctatgtctctgtgggccatgcctcccagaccaccaggagggtggcctccttcttctcccctggctctgcccagaagatccagctggtgaacaccaatggctcctggcacatcaacaggactgccctgaattgcaacgagtccatcaacactggcttctttgctgccctgttctatgtgaagaagttcaactcctctggctgctctgagaggatggcctcctgcaggcccattgacaggtttgcccagggctggggccccatcacccatgctgagtccaggtcctctgaccagaggccatactgctggcactatgccccccagccatgtggcattgtgcctgccctgcaggtctgtggccctgtctactgcttcaccccatcccctgtggtggtgggcaccactgacaggtttggcgtgcccacctacaactggggcgacaatgagactgatgtgctgctgctgaacaacaccaggcccccccagggcaactggtttggctgcacctggatgaactccactggcttcaccaagacctgtggcggccccccatgcaacattggcggcgctggcaacaacaccctgacctgccccactgactgcttcaggaagcatcctgaggccacctacaccaagtgtggctctggcccatggctgacccccaggtgcatggtggactacccatacaggctgtggcactacccatgcaccttcaacttcaccatcttcaagatcaggatgtatgtgggcggcgtggagcacaggctgaatgctgcctgcaactggaccaggggcgagaggtgcaacattgaggacagggacaggtctgagctgtcccccctgctgctgtccaccactgagtggcagatcctgccatgctccttcaccaccctgcctgccctgtccactggcctgatccatctgcatcagaacattgtggatgtgcagtacctgtacggcgtgggctccgctgtggtctccattgtgatcaagtgggagtatgtgctgctgctgttcctgctgctggctgatgcctaa

SEQ ID NO: 11 - protein, 539 aa, Hepatitis C Virus
MYEVRNVSGVYHVTNDCSNSCIVYEAADMIMHTPGCVPCVREGNSSRCWVALTPTLAARNSSIPTTTIRRHVDLLVGAAALCSAMYVGDLCGSVFLVSQLFTFSPRRYETVQDCNCSLYPGHVSGHRMAWDMMMNWSPTTALVVSQLLRIPQAVVDMVVGAHWGVLAGLAYYSMVGNWAKVLIVMLLFAGVDGTTYVSVGHASQTTRRVASFFSPGSAQKIQLVNTNGSWHINRTALNCNESINTGFFAALFYVKKFNSSGCSERMASCRPIDRFAQGWGPITHAESRSSDQRPYCWHYAPQPCGIVPALQVCGPVYCFTPSPVVVGTTDRFGVPTYNWGDNETDVLLLNNTRPPQGNWFGCTWMNSTGFTKTCGGPPCNIGGAGNNTLTCPTDCFRKHPEATYTKCGSGPWLTPRCMVDYPYRLWHYPCTFNFTIFKIRMYVGGVEHRLNAACNWTRGERCNIEDRDRSELSPLLLSTTEWQILPCSFTTLPALSTGLIHLHQNIVDVQYLYGVGSAVVSIVIKWEYVLLLFLLLADA

SEQ ID NO: 12 - DNA, 1350 bp, Artificial Sequence
  Optimized sequence encoding HCV NS5a protein
atgtctggctcctggctgagggatgtctgggactggatctgcactgtgctgactgacttcaagacctggctgcattccaagctgctgcccaggctgcctggcgacccattcttctcctgccagaggggctacaggggcgtctggaggggcgatggcgtgatgcagaccacctgcccatgtggcgcccagatcactggccatgtgaagaatggctccatgaggattgtgggccccaagacctgctccaacacctggcatggcaccttccccatcaatgcctacaccactggcccatgcaccccatcccctgcccccaactactccagggccctgtggagggtggctgctgaggagtatgtggaggtgaccagggtgggcgacttccactatgtgactggcatgaccactgacaatgtgaagtgcccatgccaggtgcctgcccctgagttcttcactgaggtggatggcgtgaggctgcacaggtatgcccctgcctgcaagcccctgctgagggatgaggtgaccttccaggtgggcctgaaccagttccctgtgggctcccagctgccatgtgagcctgagcctgatgtgactgtgctgacctccatgctgactgagccatcccacatcactgctgagactgccaagaggaggctggccaggggctcccctccatccctggcctcctcctctgcctcccagctgtctgctccatccctgaaggccacctgcaccaccaggcatgactcccctgatgctgacctgattgaggccaacctgctgtggaggcaggagatgggcggcaacatcaccagggtggagtctgagaacaaggtggtgatcctggactcctttgagcccctgagggctgaggaggatgagagggaggtctctgtggctgctgagatcctgaggaagtccaggaagttcccccctgccctgcccatctgggcgaggccatcctacaacccacccctgctggagtcctggaaggaccctgactatgtgccccctgtggtgcatggctgccccctgccccccaccatggccccacccatccccccacccaggaggaagaggactgtggtgctgactgagtccactgtctcctctgccctggctgagctggccaccaagaccttcggctcctctggctcctctgctgtggactctggcactgccacggccccccctgaccagccatctgatgatggcgacaggggctctgatgatgagtcctactcctccatgccccccctggagggcgagcctggcgaccctgacctgtctgatggctcctggtccactgtctctgaggaggcctctgaggatgtggcctgctgctcctaa

SEQ ID NO: 13 - protein, 449 aa, Hepatitis C Virus
MSGSWLRDVWDWICTVLTDFKTWLHSKLLPRLPGDPFFSCQRGYRGVWRGDGVMQTTCPCGAQITGHVKNGSMRIVGPKTCSNTWHGTFPINAYTTGPCTPSPAPNYSRALWRVAAEEYVEVTRVGDFHYVTGMTTDNVKCPCQVPAPEFFTEVDGVRLHRYAPACKPLLRDEVTFQVGLNQFPVGSQLPCEPEPDVTVLTSMLTEPSHITAETAKRRLARGSPPSLASSSASQLSAPSLKATCTTRHDSPDADLIEANLLWRQEMGGNITRVESENKVVILDSFEPLRAEEDEREVSVAAEILRKSRKFPPALPIWARPSYNPPLLESWKDPDYVPPVVHGCPLPPTMAPPIPPPRRKRTVVLTESTVSSALAELATKTFGSSGSSAVDSGTATAPPDQPSDDGDRGSDDESYSSMPPLEGEPGDPDLSDGSWSTVSEEASEDVACCS

SEQ ID NO: 14 - DNA, 1773 bp, Artificial Sequence
  Optimized sequence encoding HCV NS5b protein
atgtcctacacctggactggcgccctgatcaccccatgtgctgctgaggagtccaagctgcccatcaaccccctgtccaactccctgctgaggcatcacaacatggtctatgccaccacctccaggtctgctggcctgaggcagaagaaggtgacctttgacaggctgcatgtgcctgatgaccactacagggatgtgctgaaggagatgaaggccaaggcctccactgtgaaggcgaagctgctgtctgtggaggaggcctgcaagctgacccctccccactctgccaggtccaagtttggctatggcgccaaggatgtgaggaacctgtcctccaaggctgtgaaccacatccactctgtctggaaggacctgctggaggacactgagacccccattgacaccaccatcatggccaagaatgaggtcttctgtgtgcagcctgagaagggcggcaggaagcctgccaggctgattgtcttccctgagctgggcgtgagggtgtgtgagaagatggccctgtatgatgtggtctccaccctgccccaggctgtgatgggctcctcctatggcttccagtactcccctggccagagggtggagttcctggtgaatgcctggaagtccaagaagaaccccatgggctttgcctactgcaccaggtgctttgactccactgtgactgagtctgacatcagggtggaggagtccatctaccagtgctgtgacctggctcctgaggccaggcaggtgatcaggtccctgactgagaggctgtacattggcggccccctgaccaactccaagggccagaactgtggctacaggaggtgcagggcctctggcgtgctgaccactaactgtggcaacaccctgacctgctacctgaaggcctctgctgcttgcagggctgccaagctgcatgactgcaccatgctggtctgtggcgatgacctggtggtgatctgtgagtctgctggcacccaggaggatgctgcctccctgagggtcttcactgaggccatgaccaggtactctgccccccctggcgaccctccccagcctgagtatgacctggagctgatcacctcctgctcctccaatgtctctgtggcccatgatgcctctggcaagagggtctactacctgaccagggaccccaccacccccctggccagggctgcctgggagactgccaggcacacccctgtgaactcctggctgggcaacatcatcatgtatgcccccaccctgtgggccaggatgatcctgatgacccacttcttctccatcctgctggcccaggagcagctggagaaggccctgggctgccagatttatggcgccacctacttcattgagcccctggacctgccccagatcatccagaggctgcatggcctgtctgccttctccctgcactcctactcccctggcgagatcaacagggtggcctcctgcctgaggaagctgggcgtgccccccctgagggtgtggaggcacagggccaggtctgtgagggccaagctgctgtcccagggcggcagggctgccacctgtggcaagtacctgttcaactgggctgtgaggaccaagctgaagctgacccccatccctgctgcctcccagctggacctgtctggctggtttgtggctggctactctggcggcgacatctaccactccctgtccagggccaggcccaggtggttcatgtggtgcctgctgctgctgtctgtgggcgtgggcatctacctgctgcccaacaggtga

SEQ ID NO: 15 - protein, 590 aa, Hepatitis C Virus
MSYTWTGALITPCAAEESKLPINPLSNSLLRHHNMVYATTSRSAGLRQKKVTFDRLHVPDDHYRDVLKEMKAKASTVKAKLLSVEEACKLTPPHSARSKFGYGAKDVRNLSSKAVNHIHSVWKDLLEDTETPIDTTIMAKNEVFCVQPEKGGRKPARLIVFPELGVRVCEKMALYDVVSTLPQAVMGSSYGFQYSPGQRVEFLVNAWKSKKNPMGFAYCTRCFDSTVTESDIRVEESIYQCCDLAPEARQVIRSLTERLYIGGPLTNSKGQNCGYRRCRASGVLTTNCGNTLTCYLKASAACRAAKLHDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALGCQIYGATYFIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFVAGYSGGDIYHSLSRARPRWFMWCLLLLSVGVGIYLLPNR

SEQ ID NO: 16 - DNA, 103 bp, Artificial Sequence
  Modified Vector Sequence
cttaagcgaaggttactcttgtacctctggtacttggtcgggatggtgtagacggcgccgaagtggacgaagttcttcatttgggcccttaagatttcagctg

SEQ ID NO: 17 - DNA, 15 bp, Artificial Sequence
  Modified Vector Sequence
agatctaccatgagc

SEQ ID NO: 18 - DNA, 15 bp, Artificial Sequence
  Modified Vector Sequence
gccgaattcgcttcc

SEQ ID NO: 19 - DNA, 25 bp, Artificial Sequence
  Modified Vector Sequence
taaacccgggaattctaaagtcgac

SEQ ID NO: 20 - DNA, 12 bp, Artificial Sequence
  Modified Vector Sequence
atcaccatggat

SEQ ID NO: 21 - DNA, 15 bp, Artificial Sequence
  Modified Vector Sequence
gagatcttcatgagc

SEQ ID NO: 22 - DNA, 15 bp, Artificial Sequence
  Modified Vector Sequence
agatccaccatgcag

SEQ ID NO: 23 - DNA, 18 bp, Artificial Sequence
  Modified Vector Sequence
ggtgcagatctgatgagc

SEQ ID NO: 24 - DNA, 13 bp, Artificial Sequence
  Modified Vector Sequence
gcctaaagtcgac

SEQ ID NO: 25 - DNA, 4261 bp, Artificial Sequence
  Modified Vector Sequence
gatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctaccatgagcaccaaccccaagccccagaggaagaccaagaggaacaccaacaggaggccccaggatgtgaagttccctgggggaggccagattgtgggaggggtctacctgctgcccaggaggggccccaggctgggggtgagggctaccaggaagacctctgagaggtcccagcccaggggcaggaggcagcccatccccaaggccaggaggcctgagggccgctcctgggcccagcctggctacccctggcccctgtatggcaatgaaggctttggctgggctggctggctgctgtcccccaggggctccaggccctcctggggccccacagaccccaggaggaggtccaggaacctgggcaaggtgattgacaccctgacctgtggctttgctgacctgatgggctacatccccctggtgggggctcctgtgggaggggtggctagggctctggctcatggggtgagggtgctggaggatggggtgaactatgctactggcaacctgcctggctgctccttctccatcttcctgctggccctgctctcctgcctgacagtgcctgcttctgccgaattcgcttccaatgagaacatggagaccatgaaccagccctaccacatctgccgcggcttcacctgcttcaagaagtaaacccgggaattctaaagtcgacagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacggccgcagcggccttaattaaggccgcagcggccgtacccaggtgctgaagaattgacccggttcctcgacccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtgatcccgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcc